(19) United States
(12) Patent Application Publication
(10) Pub. No.: US 2025/0195745 A1
(43) Pub. Date: Jun. 19, 2025

(54) SHELL FOR SMALL VIAL TO FIT INTO INJECTOR OR CAP REMOVER

(71) Applicant: Janssen Biotech, Inc., Horsham, PA (US)

(72) Inventor: Ralph Fritz ZWIRNMANN, Spring House, PA (US)

(21) Appl. No.: 18/846,889

(22) PCT Filed: Feb. 23, 2023
(86) PCT No.: PCT/IB2023/051677

§ 371 Date: Sep. 13, 2024

Related U.S. Application Data
(60) Provisional application No. 63/325,354, filed on Mar. 30, 2022.

(51) Int. Cl. A61M 5/14

(52) U.S. Cl. CPC A61M 5/1408

(57) ABSTRACT

A shell that is configured to cover at least a portion of a medicant container for insertion into another device. When the shell covers the medicant container so as to define an assembled container structure, the assembled container structure may be configured to nest in a chamber, of an injector or a decapper, that the medicant container is undersized for. For example, the chamber may be configured to receive another medicant container that has a larger outer periphery than that of the medicant container covered by the shell. The assembled medicant container structure may have outer dimensions that substantially match the outer dimensions of the second medicant container. The medicant container may include a stopper that is configured to receive a needle to receive medicant from within the medicant container. The medicant container may be a vial, or a cartridge (e.g., that includes a movable plunger).

### TECHNICAL FIELD

The present invention relates to fluid containers, particularly vials and cartridges for holding medicant and dispensing medicant via an injector, and related methods.

### BACKGROUND

Some fluid containers that are configured to hold medicant have a removable cap (e.g., a plastic cap) that fixes to a lip of the respective vial (or cartridge). A cap remover with a chamber sized to receive vials (or cartridges) having the same size (e.g., a first size) can remove caps from the lips of such vials.

Some wearable medicant injectors include an injector chamber for receiving vials (or cartridges) having the same size (e.g., the first size), for example after removing the respective caps. The wearable medicant injectors can dispense metered fluid from the vial (or cartridge) in its chamber to a patient. Differently sized vials can have portions that are too big and/or too small to fit in the respective chambers of the cap removers and the wearable medicant injectors.

### SUMMARY

The present application provides for a shell that is configured to cover at least a portion of a medicant container for insertion into another device. When the shell covers the medicant container so as to define an assembled container structure, the assembled container structure may be configured to nest in a chamber, of an injector or a decapper, that the medicant container is undersized for. For example, the chamber may be configured to receive another medicant container that has a larger outer periphery than that of the medicant container covered by the shell. The assembled medicant container structure may have outer dimensions that substantially match the outer dimensions of the second medicant container. The medicant container may include a stopper that is configured to receive a needle to receive medicant from within the medicant container. The medicant container may be a vial, or a cartridge (e.g., that includes a movable plunger).

Unless otherwise specified herein, the terms “substantially equal” or “substantially match” refer to values that are the same as one another or within +/−5%, 4%, 3%, 2%, or 1% of one another.

Unless otherwise specified herein, the terms “substantially equal” or “substantially match” when used in relation to the medicant container or the assembled container structure include the respective medicant container or assembled container structure being configured to nest with a corresponding structure or configured to nest with a structure that a referenced component nests with. For example, the medicant container may be configured to nest within the shell and/or the assembled container structure may be configured to nest within the chamber, which another differently sized medicant container is configured to nest with. The assembled medicant structure being configured to nest may include at least one outer dimension of the assembled container structure having at least one outer dimension that more closely conforms to at least one corresponding inner dimension, of the respective component that the assembled container structure nests within, than the medicant container. For example, configured to nest may include at least one outer dimension of the medicant container and/or the assembled container structure being within 5%, 4%, 3%, 2%, or 1% of the at least one corresponding inner dimension of the respective component the medicant container or the assembled container structure nests within.

According to an embodiment of the present disclosure, a system comprises a device including a chamber configured to receive a first medicant container having a first size configured to nest in the chamber. The system further comprises a second medicant container having a second size that is smaller than the first size, such that the second medicant container is not configured to nest in the chamber. The system further comprises a shell configured to cover at least a portion of the second medicant container so as to define an assembled container structure that is configured to nest in the chamber.

According to another embodiment of the present disclosure, a kit comprises a first medicant container having a first size, the first medicant container containing a medication. The kit further comprises a second medicant container having a second size that is less than the first size, the second medicant container containing the medication. The kit further comprises a shell configured to receive the second medicant container so as to define an assembled container structure, whereby the shell covers at least a portion of the second medicant container. The assembled container structure has outer dimensions that are substantially equal to outer dimensions of the first medicant container structure.

According to another embodiment of the present disclosure, an assembled container structure comprises a medicant container defining a base, a side wall extending up from the base to a neck, and a medicant container interface end extending up from the neck. The medicant container contains a medication. The assembled container structure further comprises a shell configured to receive the medicant container so as to define the assembled container structure that is sized in cross-section greater than the medicant container. The shell has an internal support surface that supports the base of the medicant container, and the support surface is spaced from the base of the shell so as to define a gap therebetween. The shell extends to the neck of the vial and terminates at the neck, such that the medicant container interface end of the medicant container extends out from the shell.

### BRIEF DESCRIPTION OF THE DRAWINGS

The foregoing summary, as well as the following detailed description of illustrative embodiments of a system of the present application, will be better understood when read in conjunction with the appended drawings. For the purposes of illustrating the system of the present application, there is shown in the drawings illustrative embodiments. It should be understood, however, that the application is not limited to the precise arrangements and instrumentalities shown. In the drawings:

FIG. 1 is a front perspective view of an injection system including an injector and first medicant container disposed in a chamber of the injector;

FIG. 2 is a cross-sectional front perspective view of the injection system of FIG. 1;

FIG. 3 is a side perspective view of an assembled container structure including a vial and a shell;

FIG. 4 is an exploded perspective view of the vial and the shell of FIG. 3;

FIG. 5 is a side cross-sectional view of the assembled container structure of FIG. 3;

FIG. 6 is a side perspective cross-sectional view of an embodiment of the injection system including the injector of FIG. 1 and the assembled container structure of FIG. 3 disposed within the chamber;

FIG. 7 is a side perspective view of another embodiment of the assembled container structure, which includes a shell that does not include a window for viewing a main body of the vial;

FIG. 8 is a partial side perspective view of the assembled container structure of FIG. 7, in which one piece of the shell is removed from the vial;

FIG. 9 is a side perspective view of one piece of the shell of FIG. 7, in which the piece defines a ledge with a radially inwardly facing concave surface;

FIG. 10 is a side cross-sectional view of the assembled container structure of FIG. 7;

FIG. 11 is a side perspective view of another embodiment of the shell, in which the shell that does not include a window for viewing a main body of the vial and the shell defines a ledge that does not form a radially inwardly facing concave surface;

FIG. 12 is a side perspective exploded view of another embodiment of the assembled container structure, in which the shell includes a pair of circumferentially extending protrusions that are configured to be received in respective circumferentially facing cavities to lock the shell in an assembled configuration;

FIG. 13 is a side cross-sectional view of the assembled container structure of FIG. 12, in the assembled configuration;

FIG. 14 is another side cross-sectional view of the assembled container structure of FIG. 13;

FIG. 15 is a side perspective exploded view of another embodiment of the shell, in which the shell includes a plurality of radially inwardly extending flexible fingers such that the shell is configured to selectively receive medicant containers that have various different sizes;

FIG. 16 is a side perspective view of another embodiment of the shell, in which the shell is formed as a single piece with a living hinge that provides for pivoting of one part of the shell relative to another;

FIG. 17 is a side perspective exploded view of another embodiment of the assembled container structure, which includes a cartridge and another embodiment of the shell, which does not include a ledge;

FIG. 18 is a side cross-sectional view of the assembled container structure of FIG. 17, in which the shell includes a longitudinally extending wall that secures the cartridge relative to the shell;

FIG. 19 is a side perspective exploded view of another embodiment of the assembled container structure, which includes a cartridge and another embodiment of the shell, which forms a radially outer wall that extends circumferentially around a lip of the cartridge;

FIG. 20 is a side cross-sectional view of the assembled container structure of FIG. 19, in which the shell includes the radially outer wall that extends circumferentially around the lip of the cartridge;

FIG. 21 is a side cross-sectional view of a decapping system including a container decapper and the assembled container structure of FIG. 3 being lowered into a chamber of the container for removal of a cap from the assembled container structure;

FIG. 22 is a side cross-sectional view of the decapping system of FIG. 21, in which the assembled container structure is nested in the chamber;

FIG. 23 is a side cross-sectional view of the decapping system of FIG. 22, in which the cap is removed from the assembled container structure while the assembled container structure is nested in the chamber;

FIG. 24 is a perspective view of a portion of another embodiment of a decapping system, which includes a plurality of chambers that are each configured to receive the first medicant container or the assembled container structure of FIG. 3 having substantially the same size as the first medicant container; and

FIG. 25 is a side view of a portion of the decapping system of FIG. 24, including a portion of a fixed member with a ramped surface configured to remove a cap from the first medicant container and the assembled container structure.

### DETAILED DESCRIPTION OF ILLUSTRATIVE EMBODIMENTS

The present disclosure can be understood more readily by reference to the following detailed description taken in connection with the accompanying figures and examples, which form a part of this disclosure. It is to be understood that this disclosure is not limited to the specific devices, methods, applications, conditions or parameters described and/or shown herein, and that the terminology used herein is for the purpose of describing particular embodiments by way of example only and is not intended to be limiting of the scope of the present disclosure. Also, as used in the specification including the appended claims, the singular forms “a,” “an,” and “the” include the plural, and reference to a particular numerical value includes at least that particular value, unless the context clearly dictates otherwise.

The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

Referring to FIG. 1, an injection system 30 (an example of a system) may include an injector 32 (an example of a device) and a first medicant container 34 disposed in a chamber 36 of the injector.

The injector 32 (e.g., a patch pump) is configured to inject medication contained in the first medicant container 34 into a patient. For example, the injector 32 includes an injection needle 38 that is configured to dispense medicant contained in the first medicant container 34 to a patient, as discussed further below.

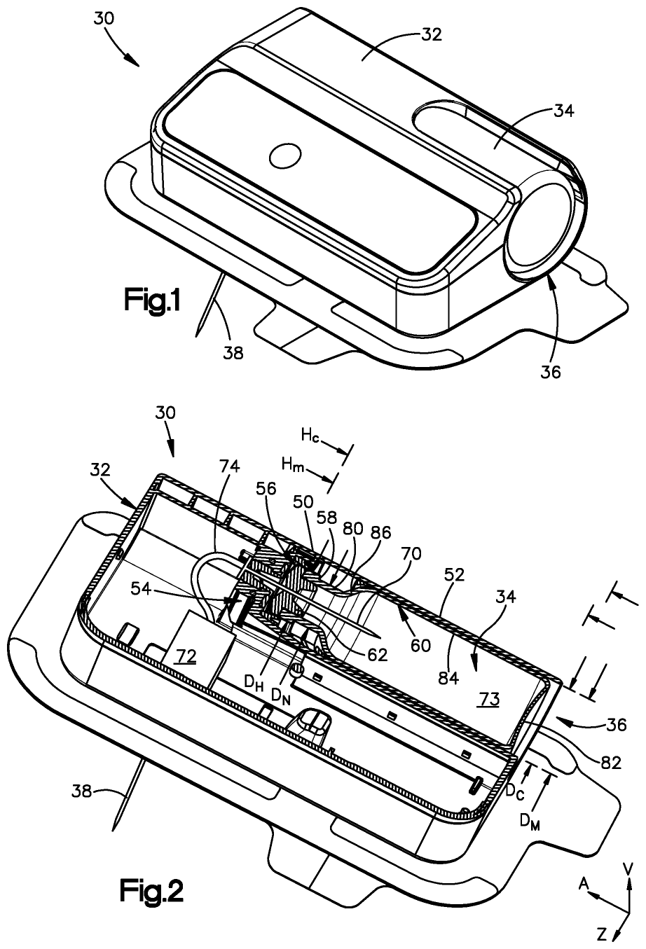

Turning to FIG. 2, the chamber 36 may define a space configured to receive the first medicant container 34. For example, the chamber 36 may be configured to receive a size 2R, 4R, 6R, 8R, 10R, 15R, 20R, 25R, or 30R first medicant container 34, in accordance with International Organization for Standardization (ISO) 8362-1, as most recently available as of Jan. 1, 2022, and which is incorporated by reference herein. In some embodiments, the first medicant container is a vial. In an embodiment, the first medicant container is a cartridge.

For example, the chamber 36 may be configured to receive the first medicant container 34 having one of the above sizes such that the first medicant container 34 nests in the chamber 36 for injection by the injector 32. The chamber 36 may extend along a longitudinal axis A, that is perpendicular to both a lateral axis V and a transverse axis Z, each of which are perpendicular to one another.

For example, the injector 32 may include a housing 50 that defines a main cylindrical portion 52 that has an inner diameter D_{C} (an example of a cross-sectional dimension) that is substantially equal to a corresponding portion of the first medicant container 34 such that the main cylindrical portion 52 abuts the first medicant container 34.

The injector 32 may include a mechanical interface 54 that is configured to receive a head 56 of the first medicant container 34. For example, the mechanical interface 54 may define cylindrical opening that is configured to hold the head 56. The mechanical interface 54 may be flexible (e.g., a flexible rubber gasket) such that the cylindrical opening is configured to widen to accommodate and secure the head 56.

The head 56 may include a lip 58, defined by a main body 60 of the first medicant container 34, and a head gasket 62. In some embodiments, the head includes a cover that is crimped over the head gasket and the lip to secure the head gasket to the lip while allowing insertion of a needle into the head gasket, as discussed below.

As discussed below in relation to decapping, in some embodiments, a cap attaches to the head to protect the head gasket prior to insertion into the injector. Such a cap can be removed to access the head gasket.

The injector 32 may include an inlet needle 70 (e.g., a cannulated inlet needle) fluidly connected to a pump 72 that is fluidly connected to the injection needle 38. A fluid line 74 may fluidly connect the inlet needle 70 to an inlet of the pump 72. The injection needle 38 may be fluidly connected directly to an outlet of the pump 72. In an embodiment, the injection needle is fluidly connected to an outlet of the pump.

The inlet needle 70 may be configured to be inserted into and through the head 56 into a chamber 73 of the first medicant container 34 to reach the medication held by the first medicant container 34 in the chamber 73. For example, the chamber 73 has a fixed volume for holding the medication.

During an injection, the pump 72 may suction medication from the inlet needle 70 via the fluid line 74, and output the medication to the injection needle 38 for injection into the patient. The pump 72 may be configured to inject a predetermined amount of medication, for example, based on a user's input. In an embodiment, the injector includes a third needle that is inserted into the head of the first medicant container and fluidly connected to atmosphere to equalize pressure within the first medicant container and atmosphere.

The main body 60 may define a neck 80, a base 82, a side wall 84, and a radially inwardly extending shoulder 86 that extends from the sidewall 84 to the neck 80. The neck 80 may extend between the head 56 and the radially inwardly extending shoulder 86 along the longitudinal axis A. For example, the sidewall 84 and the neck 80 may each define a respective cylindrical portion that extends along the longitudinal axis from respective ends of the radially inwardly extending shoulder 86.

The neck 80 may define a neck diameter D_{N} (an example of a cross-sectional dimension). For example, the neck diameter D_{N} may be any one of 13 millimeters (mm) or 20 mm. In some embodiments, the neck diameter D_{N} is anyone of 10.5 mm, 16.5 mm, or 17.5 mm.

The head 56 may define a medicant container interface end that is configured to be received by the corresponding mechanical interface 54 of the injector 32. The head 56 (e.g., the medicant container interface) may define a head diameter D_{H} (an example of a cross-sectional dimension) that is substantially equal to a diameter of the cylindrical opening of the mechanical interface 54. For example, the head diameter D_{H} may be any one of 13 mm or 20 mm. In an embodiment, the diameter of the cylindrical opening is the same as the head diameter D_{H} or within +/−5%, 4%, 3%, 2%, or 1% of the head diameter D_{H}. In an embodiment, the head includes a cap (e.g., an aluminum cap) that defines a head diameter D_{H} of anywhere from 13.3 mm to 13.5 mm or anywhere from 20.3 mm to 20.5 mm.

The side wall 84 may extend between the base 82 and the radially inwardly extending shoulder 86 along the longitudinal axis A. For example, the side wall 84 may define a cylindrical portion with a main body diameter D_{M} that is substantially equal to the chamber diameter D_{C}. The main body diameter D_{M} may be any one of 16 mm, 22 mm, 24 mm, or 30 mm. The chamber diameter D_{C} may be the same as the main body diameter D_{M} or within +/−5%, 4%, 3%, 2%, or 1% of the main body diameter D_{M}.

The entire first medicant container 34 may define a medicant container height H_{M} that is substantially equal to a chamber height H_{C} of the chamber 36. For example, the medicant container height H_{M} may be any one of 35 mm, 40 mm, 45 mm, 55 mm, 60 mm, 65 mm, or 75 mm. The chamber height H_{C} may be the same as the medicant container height or within +/−5%, 4%, 3%, 2%, or 1% of the medicant container height H_{M}.

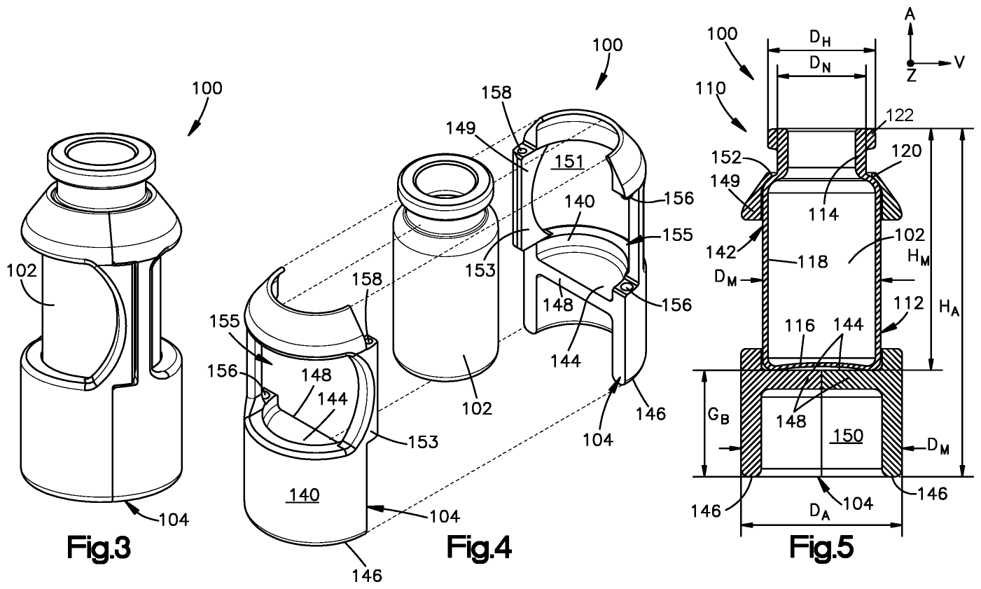

Turning to FIGS. 3 to 5, an example of an assembled container structure is illustrated at 100. The assembled container structure 100 may include a second medicant container 102 (e.g., a vial with a fixed volume chamber) and a shell 104 such that the assembled container structure 100 is configured to nest in the chamber 36. The second medicant container 102, the shell 104, and the first medicant container 34 discussed above may together form at least a portion of a kit.

In some embodiments, the shell 104 and a second shell may be provided as part of a kit. For example, the second shell may be similar to the shell 104, but with at least one different inner dimension or at least one different outer dimension. Both the shell 104 and the second shell may have outer dimensions greater than those defined by size 2R of ISO 8362-1. Both the shell 104 and the second shell may have inner dimensions that are less than those defined by size 30R of ISO 8362-1.

The second medicant container 102 may be substantially the same as the first medicant container 34, except a height and/or cross-sectional dimension may be less than that of the first medicant container 34. The second medicant container 102 may be undersized such that the second medicant container 102 could be subject to inadvertent movement relative to the injector 32 that could cause the second medicant container 102 to become displaced relative to the injection needle 38. For example, the second medicant container 102 may be configured such that the second medicant container 102 is not configured to nest in the chamber 36.

Referring in particular to FIG. 5, the second medicant container 102 may include a head 110 and a main body 112. The main body 112 may define a neck 114, a base 116, a side wall 118, and a radially inwardly extending shoulder 120 that extends from the sidewall 118 to the neck 114. The neck 114 may extend between the head 110 and the radially inwardly extending shoulder 120 along the longitudinal axis A. For example, the sidewall 118 and the neck 114 may each define a respective cylindrical portion that extends along the longitudinal axis from respective ends of the radially inwardly extending shoulder 120.

The head 110 may define a medicant container interface end that is configured to be received by the corresponding mechanical interface 54 of the injector 32. In another embodiment, the medicant container interface end is undersized for the mechanical interface 54.

The head 110 (e.g., the medicant container interface) may define a head diameter D_{H} (an example of a cross-sectional dimension) that is substantially equal to a diameter of the cylindrical opening of the mechanical interface 54. For example, the head diameter D_{H} may be any one of 13 mm or 20 mm. In an embodiment, the diameter of the cylindrical opening is the same as the head diameter D_{H} or within +/−5%, 4%, 3%, 2%, or 1% of the head diameter D_{H}. In some embodiments, the head diameter is less than the diameter of the cylindrical opening such that the head is too small to nest in the mechanical interface 54. In an embodiment, the head includes a cap (e.g., an aluminum cap) that defines a head diameter D_{H} of anywhere from 13.3 mm to 13.5 mm or anywhere from 20.3 mm to 20.5 mm.

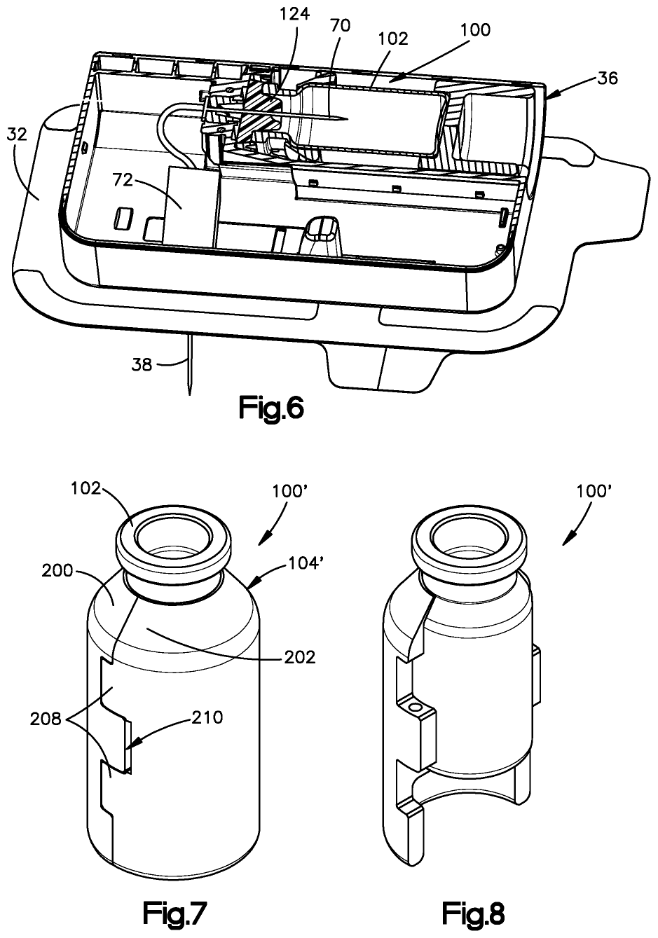

The head 110 may include a lip 122 that may be at least partially defined by the main body 112. In an embodiment, the head includes a head gasket 124 (see e.g., FIG. 6) substantially the same as the head gasket 62. In some embodiments, the head includes a cover that is crimped over the head gasket and the lip to secure the head gasket to the lip while allowing insertion of a needle into the head gasket.

The neck 114 may define a neck diameter D_{N} (an example of a cross-sectional dimension). For example, the neck diameter D_{N} may be any one of 13 mm or 20 mm. In some embodiments, the neck diameter D_{N} is anyone of 10.5 mm, 16.5 mm, or 17.5 mm.

The sidewall 118 may extend between the base 116 and the radially inwardly extending shoulder 120 along the longitudinal axis A. For example, the side wall 118 may define a cylindrical portion with a main body diameter D_{M} that is less than the chamber diameter D_{C}. The main body diameter D_{M} may be any one of 16 mm, 22 mm, 24 mm, or 30 mm. The chamber diameter D_{C} may be at least one of 10%, 20%, 30%, 40%, 50%, 100%, 150%, 200%, or 300% greater than the main body diameter D_{M}.

The entire second medicant container 102 may define a medicant container height H_{M} that is less than the chamber height H_{C} of the chamber 36. For example, the medicant container height H_{M} may be any one of 35 mm, 40 mm, 45 mm, 55 mm, 60 mm, 65 mm, or 75 mm. The chamber height H_{C} may be the same as the medicant container height or within +/−5%, 4%, 3%, 2%, or 1% of the medicant container height H_{M}. The chamber height H_{C} may be at least one of 10%, 20%, 30%, 40%, 50%, 100%, 150%, 200%, or 300% greater than the medicant container height H_{M}. In an embodiment, the second medicant container includes a head gasket, and the medicant container height extends from a top of the head gasket to the bottom of the base of the second medicant container. For example, FIG. 6 illustrates an example of a second medicant container with a head gasket.

In an embodiment, the head of the second medicant container includes a head gasket and a cover that secures the head gasket to a lip of the second medicant container. For example, the head of the second medicant container may be substantially identical to the head of the first medicant container. In some embodiments, the head of the second medicant container may be smaller than the head of the first medicant container.

The shell 104 may be configured to cover at least a portion of the second medicant container 102 so as to define the assembled container structure 100. A cross-sectional dimension and height of the assembled container structure may be greater than those of the second medicant container 102. For example, the assembled container structure 100 may define a main structure body diameter D_{A} that is greater than the main body diameter D_{M} of the second medicant container 102. The main structure body diameter D_{A} may be at least one of 10%, 20%, 30%, 40%, 50%, 100%, 150%, 200%, or 300% greater than the main body diameter D_{M} of the second medicant container 102.

For example, the main structure body diameter D_{A} may be any one of 16 mm, 22 mm, 24 mm, or 30 mm. The main structure body diameter D_{A} may be the same as the chamber diameter D_{C} and the main body diameter D_{M} of the first medicant container 34, or within +/−5%, 4%, 3%, 2%, or 1% of the chamber diameter D_{C} and the main body diameter D_{M} of the first medicant container 34.

The structure height H_{A} may be at least one of 10%, 20%, 30%, 40%, 50%, 100%, 150%, 200%, or 300% greater than the medicant container height H_{M} of the second medicant container 102.

The structure height H_{A} may be any one of 35 mm, 40 mm, 45 mm, 55 mm, 60 mm, 65 mm, or 75 mm. The structure height H_{A} may be the same as the chamber height H_{C} and the medicant container height H_{M} of the first medicant container 34, or within +/−5%, 4%, 3%, 2%, or 1% of the chamber height H_{C} and the medicant container height H_{M} of the first medicant container 34.

The neck diameter D_{N} defined by the neck 114 may define the neck diameter of the assembled container structure 100. In some other embodiments, the shell defines the neck diameter of the assembled container structure (e.g., as discussed below with reference to FIGS. 17-20).

Referring now to FIGS. 4 and 5, the shell 104 has at least one outer dimension that corresponds to a first size in a standard (e.g., (ISO) 8362-1), and an internal dimension that corresponds to a second size of the standard that is smaller than the first size. The at least one outer dimension is sized to be received in the chamber 36 of the injector 32 (FIG. 2), the chamber 36 having at least one inner dimension that corresponds to the first size defined by the standard. For example, the at least one outer dimension of the shell 104 may be a body diameter, a neck diameter, a head diameter, and/or a length that corresponds with a respective body diameter, neck diameter, head diameter, and/or a length of the chamber 36. Thus, the at least one outer dimension of the shell conforms to the at least one inner dimension of the chamber 36.

The at least one inner dimension of the shell 104 may define a portion of a receptacle 142 that is sized receive the medicant container 102, where the container has the at least one an outer dimension that corresponds to the second size defined by the standard. For example, the at least one inner dimension of the receptacle 142 of the shell 104 may be a body diameter, a neck diameter, a head diameter, and/or a length that corresponds with a respective body diameter, neck diameter, head diameter, and/or a length of the medicant container 102. Thus, the shell can be an adapter that is configured to adapt the second medicant container 102 having the second size to the chamber 36 of a device (e.g., the injector 32) having the first size.

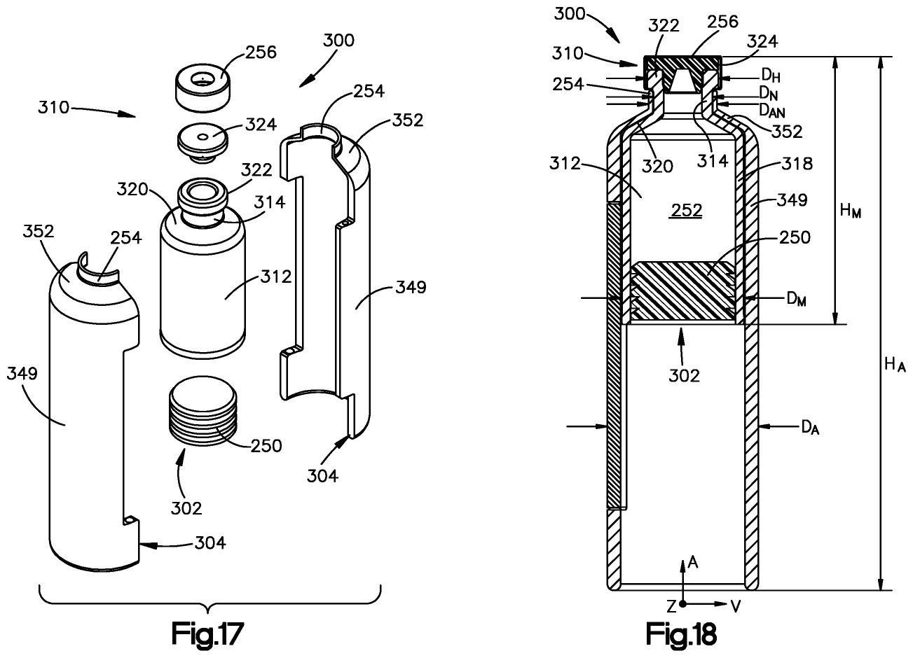

For example, the shell 104 may include a pair of interlocking bodies 140 that when interlocked together define receptacle 142 that is configured to hold the second medicant container 102 relative to the shell 104. The interlocking bodies 140 may be interlocked together by moving the interlocking bodies 140 together along the illustrated dashed lines extending between the interlocking bodies 140. The assembled container structure 100 is defined by the second medicant container 102 and the shell 104 when the receptacle 142 holds the second medicant container 102. In some other embodiments, the shell locks onto the medicant container in another suitable manner. For example, as discussed below with reference to FIG. 16, the shell may be formed by a single body that is configured to lock in a closed position to lock onto the medicant container. In another embodiment, the shell may be biased into the closed position. In other alternative embodiments, the shell does not does not include multiple bodies that are separated by a longitudinal split and instead an opening of the shell (e.g., at the top end of the shell) is configured to stretch to receive the second medicant container in the receptacle (e.g., in a manner similar to a sock), thereby locking the second medicant container in the receptacle.

The receptacle may define a diameter that is identical to the main body diameter D_{M} of the second medicant container 102. In an embodiment, the diameter defined by the receptacle is within +/−5%, 4%, 3%, 2%, or 1% of the main body diameter D_{M} of the second medicant container 102.

Each of the interlocking bodies 140 may be identical to one another. For example, each interlocking body 140 may define a portion 144 of an internal support surface (an example of a floor) that is configured to support the base 116 of the second medicant container 104. Each portion 144 of the internal support surface may be defined by a respective radially extending wall portion 148 of each interlocking body 140. In some other embodiments, the interlocking bodies are not identical to one another. For example, only one of the interlocking bodies may define the entire internal support surface.

The interlocking bodies may together define a sidewall 149 of the shell 104, which may define a sidewall of the assembled container structure 100. Each interlocking body 140 may define a window 151 in the sidewall 149 such that the user can see the second medicant container 102 through the window 151. For example, each window 151 is a radially extending through hole in the sidewall 149. In some embodiments, the sidewall includes at least a portion that is transparent or translucent such that the user can see the second medicant container through the transparent or translucent portion. In some embodiments, the sidewall has a translucent portion that is colored or tinted such that the user is unable to determine the color of the second medicant container or the medicant through the translucent portion.

In some embodiments, the shell does not include a window (e.g., as illustrated in FIG. 7) and at least a portion of the shell is colored, tinted, or opaque such that the user is unable to determine the color of the medicant through the shell. For example, the entire shell may be colored, tinted, or opaque. During a blinded clinical trial a colored, tinted, or opaque shell may hide a color of a medicant (e.g., hide the color of the medicant from a trial researcher and a trial subject).

The shell 104 may include at least one fastener or coupler that fastens or locks the bodies 140 to one another. For example, as shown in FIG. 4, each sidewall 149 may define a circumferentially extending locking arm 153 and a corresponding arm receiving cavity 155 that is configured to receive and lock with the circumferentially extending locking arm 153 of the other side wall 149. For example, each arm receiving cavity 155 may at respective longitudinal ends define a pair of longitudinally extending bumps 156 that longitudinally face one another. Each circumferentially extending locking arm 153 may at respective longitudinal ends define a pair of longitudinally facing divots 158 that face away from one another. The longitudinally facing divots 158 may be configured to receive the bumps 156 when the respective locking arm 153 is received by the corresponding arm receiving cavity 155 such that that locking arm 153 is secured within the arm receiving cavity 155.

In some other embodiments, the interlocking bodies may be fastened or locked together in another suitable manner. For example, the shell may include a bolt or other fastener that is configured to couple adjacent bodies 140 to one another. Alternatively, the shell may include a toothed member on one of the bodies 140 that is configured to be received by an opening of the other one of the bodies 140.

Referring to only FIG. 5, each interlocking body 140 may define a base member 146 that together define a base that is longitudinally spaced from each portion 144 of the support surface such that a longitudinal gap G_{B} is defined therebetween.

The base may define a cavity 150 that extends longitudinally toward the inner support surface. In some embodiments, the base does not define the cavity.

Each interlocking body 140 may extend longitudinally to the neck 114 and terminate at the neck 114, such that the medicant container interface end of the second medicant container 104 extends out from the shell 104. For example, the interlocking bodies 140 may together define a radially inwardly extending rim 152 that is configured to abut the shoulder 120. The radially inwardly extending rim 152 may abut a portion of the neck 114.

The radially inwardly extending rim 152 may be longitudinally offset from the internal support surface of the shell 104 such that the second medicant container 102 is held therebetween. For example, each portion 144 of the internal support surface may abut the base 116 of the second medicant container 102 and the radially inwardly extending rim 152 may abut the shoulder 120 of the second medicant container 102.

Turning to FIG. 6, the injector 32 is shown in combination with the assembled container structure 100 nested in the chamber 36 such that the injector 32 is configured to inject the contents of the second medicant container 102. The assembled container structure 100 may include the gasket 124, as discussed above.

Injecting medicant from the assembled container structure 100 may be substantially the same as with the first medicant container 34. For example, during an injection, the pump 72 may suction medication from the inlet needle 70 in the second medicant container 102 via the fluid line 74, and output the medication to the injection needle 38 for injection into the patient. The pump 72 may be configured to inject a predetermined amount of medication, for example, based on the user's input. In an embodiment, the injector includes a third needle that is inserted into the head of the second medicant container and fluidly connected to atmosphere to equalize pressure within the first medicant container and atmosphere.

Referring now to FIGS. 7-10, a second embodiment of the assembled container structure is shown. It is to be appreciated that the second embodiment can be similar to the first embodiment of the assembled container structure shown in FIGS. 3-5. Accordingly, the same reference numbers used above with reference to features of the first embodiment can be also used with a “prime” notation in reference to similar features of the second embodiment. It is also to be appreciated that, unless otherwise set forth below, the components (and features thereof) of the assembled container structure 100′ of the second embodiment can be similar to those of the first embodiment.

Referring initially to FIG. 7, the assembled container structure 100′ includes the second medicant container 102 and a shell 104′. The shell 104′ may not define a window that a sidewall of the second medicant container 102 can be seen through.

The shell 104′ may include a pair of interlocking bodies 200 and 202. The interlocking bodies 200 and 202 may be different from one another.

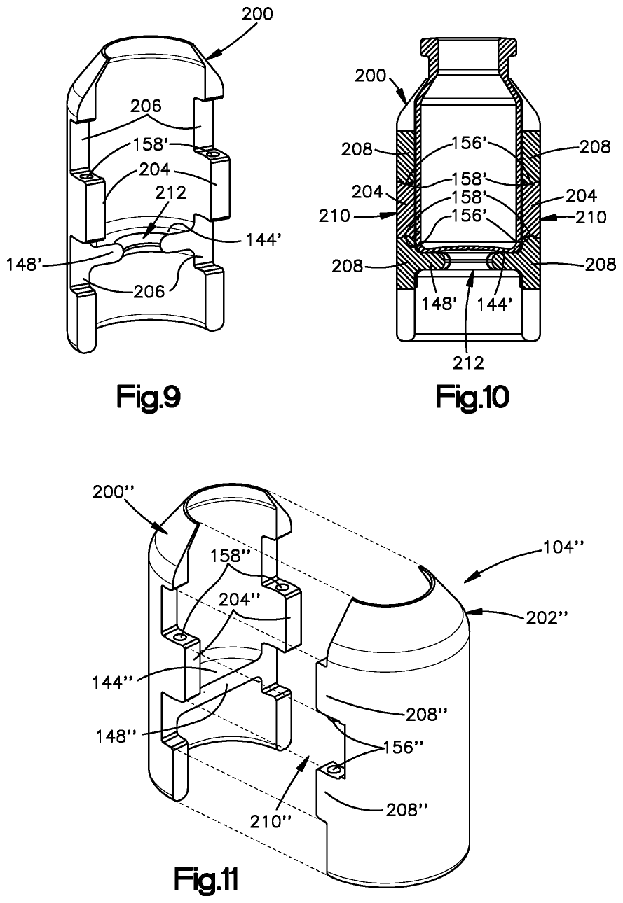

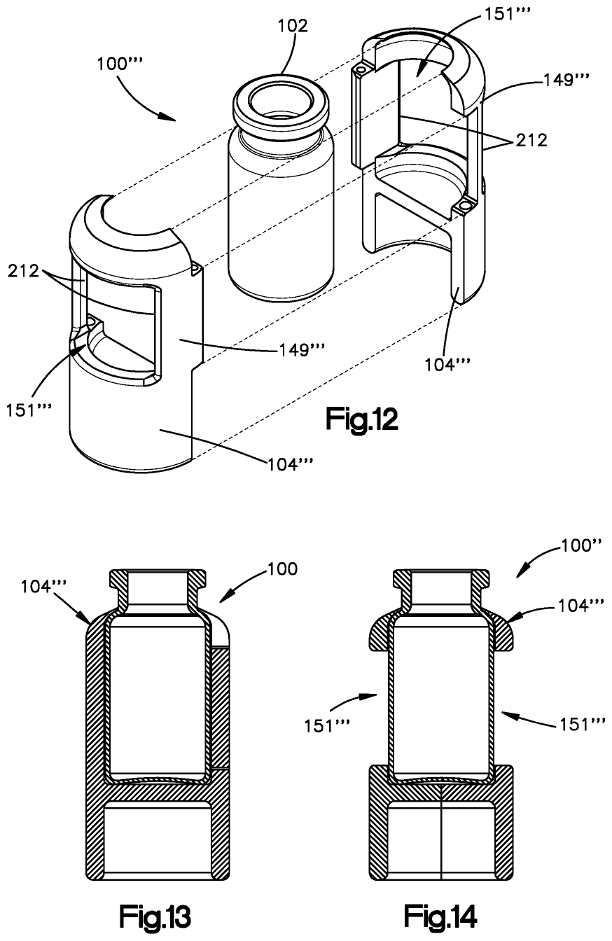

For example, with reference to FIGS. 9 and 10, the interlocking body 200 may at each of its sides define a circumferentially extending arm 204 and a pair of locking arm receiving cavities 206. The interlocking body 202 may at each of its sides define a pair of circumferentially extending locking arms 208 that together define a locking cavity 210. For example, each pair of locking arms 208 define a respecting pair of bumps 156′ that longitudinally face one another. The arms 204 each define a pair of divots 158′ that are configured to receive the bumps 156′ when the respective arm 204 is received by the corresponding locking cavity 210 such that that each arm 204 is secured within the locking cavity 210.

Turning to FIGS. 9 and 10, each interlocking body 200 and 202 may define a portion 144′ of an internal support surface that is defined by a respective radially extending wall portion 148′. The radially extending wall portion may include a central through hole. For example, the through hole may provide for the user to view a bottom of the second medicant container 102.

Referring now to FIG. 11, a third embodiment of the shell is shown. It is to be appreciated that the third embodiment can be similar to the second embodiment of the shell shown in FIGS. 7-10. Accordingly, the same reference numbers used above with reference to features of the first embodiment can be also used with a “double prime” notation in reference similar features of the third embodiment. It is also to be appreciated that, unless otherwise set forth below, the components (and features thereof) of the shell 104″ of the third embodiment can be similar to those of the first and second embodiments.

The shell 104″ may include a pair of interlocking bodies 200″ and 202″. The interlocking bodies 200″ and 202″ may be different from one another.

For example, the interlocking body 200″ may at each of its sides define a circumferentially extending arm 204″. The interlocking body 202″ may at each of its sides define a pair of circumferentially extending locking arms 208″ that together define a locking cavity 210″. Each pair of locking arms 208 define a respecting pair of bumps 156″ that longitudinally face one another. The arms 204″ each define a pair of divots 158″ that are configured to receive the bumps 156″ when the respective arm 204″ is received by the corresponding locking cavity 210″ such that that each arm 204″ is secured within the locking cavity 210″.

Each interlocking body 200″ and 202″ may define a portion 144″ of an internal support surface that is defined by a respective radially extending wall portion 148″. The radially extending wall portion may not include a through hole. For example, each portion 144″ may have a semi-circular shape.

Referring now to FIGS. 12-14, another embodiment of the assembled container structure is shown. It is to be appreciated that this embodiment can be similar to the second embodiment of the assembled container structure shown in FIGS. 7-10. Accordingly, the same reference numbers used above with reference to features of the first embodiment can be also used with a “triple prime” notation in reference to similar features of the fourth embodiment. It is also to be appreciated that, unless otherwise set forth below, the components (and features thereof) of the assembled container structure 100″′ of this embodiment can be similar to those of the first and second embodiments.

The assembled container structure 100″′ includes the second medicant container 102 and a shell 104″′. As shown in FIGS. 12 and 14, the shell 104′ may define a pair of radially opposed windows 151″′.

Each window 151″′ may be a radially extending through hole in a sidewall 149″′ of the shell 104″′. For example, the through hole may be defined by circumferentially facing sides 220 of the side wall 149″′. The circumferentially facing sides 220 may be planar and extend longitudinally such that the circumferentially facing sides are parallel to one another. When the second medicant container 102 is viewed through the through hole, the through hole may appear to the viewer to have straight sides that extend longitudinally.

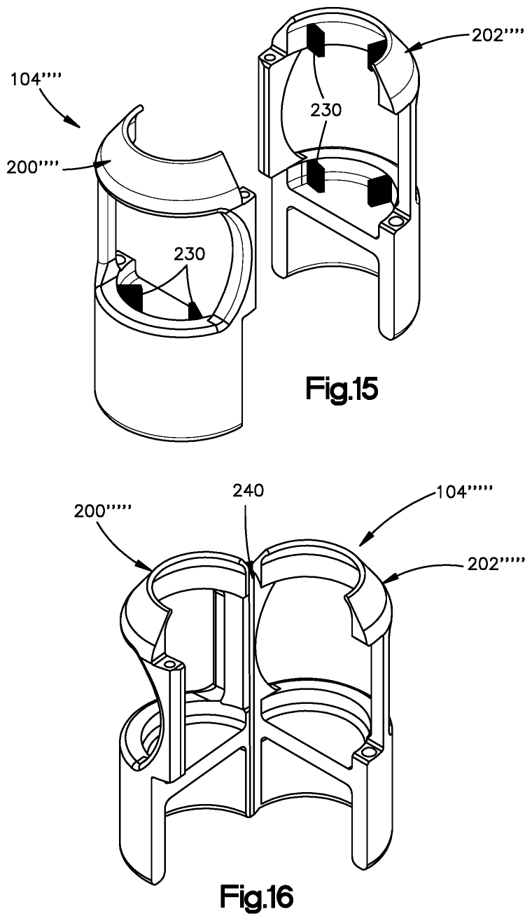

Referring now to FIG. 15, a fifth embodiment of the shell is shown. It is to be appreciated that the fifth embodiment can be similar to the other embodiments of the shell shown in FIGS. 3-13. Accordingly, the same reference numbers used above with reference to features of the other embodiments can be also used with a “quadruple prime” notation in reference to similar features of the fifth embodiment. It is also to be appreciated that, unless otherwise set forth below, the components (and features thereof) of the shell 104″″ of this embodiment can be similar to those of the other embodiments.

The shell 104″″ may include a pair of interlocking bodies 200″″ and 202″″. The interlocking bodies 200″″ and 202″″ may be configured to together selectively hold the second medicant container 102 and a differently sized medicant container. For example, the shell may include a plurality of flexible fingers 230 that are each configured to flex to selectively hold the second medicant container 102 and the differently sized medicant container. The flexible fingers 230 may be comprised of rubber.

The plurality of fingers 230 may be symmetrically spaced circumferentially around the interior of the shell 104″″. The plurality of fingers 230 may include multiple rows of fingers 230 that are longitudinally spaced from one another. In an embodiment, eight or fewer fingers are provided in the interior of the shell. In an embodiment, more than eight fingers are provided in the interior of the shell.

The differently sized medicant container may have a different cross-sectional dimension from the second medicant container 102. For example, the differently sized medicant container may be substantially the same as the second medicant container 102, but with a different cross-sectional dimension (e.g., as described above in relation to the second medicant container 102) from the second medicant container 102.

The third medicant container may have a height different from the second medicant container 102. For example, the differently sized medicant container may be substantially the same as the second medicant container 102, but with a different height (e.g., as described above in relation to the second medicant container 102) from the second medicant container 102. In an embodiment, the differently sized medicant container has a different height and a different cross-sectional dimension compared to the second medicant container 102.

Referring now to FIG. 16, a sixth embodiment of the shell is shown. It is to be appreciated that the sixth embodiment can be similar to the other embodiments of the shell shown in FIGS. 3-15. Accordingly, the same reference numbers used above with reference to features of the other embodiments can be also used with a “quadruple prime” notation in reference to similar features of the sixth embodiment. It is also to be appreciated that, unless otherwise set forth below, the components (and features thereof) of the shell 104″″′ of this embodiment can be similar to those of the other embodiments.

The shell 104″″′ may include a pair of interlocking bodies 200″″′ and 202″″′. The interlocking bodies 200″″′ and 202″″′ may be formed as a single piece. For example, the interlocking bodies 200″″′ and 202″″′ may include a living hinge 240 that provides for the interlocking bodies 200″″′ and 202″″′ to be pivoted relative to one another. Moreover, the living hinge 240 provides for the interlocking bodies 200″″′ and 202″″′ to be pivotable between an unlocked configuration exemplified in Fig. and a locked configuration (e.g., similar to the configuration shown in FIG. 3).

Referring now to FIGS. 17 and 18, another embodiment of the assembled container structure is shown. It is to be appreciated that this embodiment can be similar to the other embodiments of the assembled container structure shown, for example, in FIGS. 3-8. Accordingly, the same reference numbers used above with reference to the other embodiments can be also used and indexed by 200 in reference to this embodiment. It is also to be appreciated that, unless otherwise set forth below, the components (and features thereof) of the assembled container structure 300 of this embodiment can be similar to those of the other embodiments.

The assembled container structure 300 may include a second medicant container 302 and a shell 304. The second medicant container 302 may be a cartridge that includes a head 310, a cartridge body 312, and a plunger 250.

With reference to FIG. 18, the plunger 250 may be configured to define a portion of a variable volume chamber 252. For example, the variable volume chamber 252 may be defined by the plunger 250, the cartridge body 312, and a head gasket 324. The plunger 250 may be configured to move longitudinally relative to the cartridge body 312. For example, when medicant is dispensed from the variable volume chamber 252, the plunger 250 may move longitudinally toward the head 310 to equalize pressure within the variable volume chamber with atmosphere. In an embodiment, an actuator (e.g., an electromechanical actuator) moves the plunger to cause dispensing by increasing pressure within the variable volume chamber.

The cartridge body 312 defines a neck 314, a side wall 318, and a radially inwardly extending shoulder 320 that extends from the sidewall 318 to the neck 314.

The head 310 may define a medicant container interface end that is configured to be received by the corresponding mechanical interface 54 of the injector 32.

The head (e.g., the medicant container interface) may define a head diameter D_{H} (an example of a cross-sectional dimension) that is substantially equal to a diameter of the cylindrical opening of the mechanical interface 54. For example, the head diameter D_{H} may be any one of 7.5 mm, 13 mm, or 20 mm. In an embodiment, diameter of the cylindrical opening is the same as the head diameter D_{H} or within +/−5%, 4%, 3%, 2%, or 1% of the head diameter D_{H}. In an embodiment, the head includes a cap (e.g., an aluminum cap) that defines a head diameter D_{H} of anywhere from 7.8 mm to 8.0 mm, anywhere from 13.3 mm to 13.5 mm, or anywhere from 20.3 mm to 20.5 mm.

The neck 314 may define a neck diameter D_{N} that is less than the neck diameter D_{N} of the first medicant container 34. For example, the neck diameter D_{N} of the first second medicant container 34 may be at least one of 10%, 20%, 30%, 40%, 50%, 100%, 150%, 200%, or 300% greater than the neck diameter D_{N} of the second medicant container 31.

The shell 304 includes a wall 254 that extends along the longitudinal axis A from a radially inwardly extending rim 352, which extends longitudinally from a side wall 349. The wall 254 may be configured to radially abut the neck 314 and extend between a lip 322 and a radially inwardly extending shoulder 320 of the cartridge body 312. For example, the wall 254 may longitudinally abut the head 310 and the radially inwardly extending shoulder 320, such that the second medicant container 302 is secured to and nested within the shell 304.

The entire second medicant container 302 may define a medicant container height H_{M} that is less than the chamber height H_{C} of the chamber 36. For example, the medicant container height H_{M} may be any one of 35 mm, 40 mm, 45 mm, 55 mm, 60 mm, 65 mm, or 75 mm. The chamber height H_{C} may be the same as the medicant container height or within +/−5%, 4%, 3%, 2%, or 1% of the medicant container height H_{M}. The chamber height H_{C} may be at least one of 10%, 20%, 30%, 40%, 50%, 100%, 150%, 200%, or 300% greater than the medicant container height H_{M}.

The shell 304 may be configured to cover at least a portion of the second medicant container 302 so as to define the assembled container structure 300. A cross-sectional dimension and height of the assembled container structure may be greater than those of the second medicant container 302. For example, the assembled container structure 300 may define a structure neck diameter D_{AN} that is greater than the neck diameter D_{N} of the second medicant container 302. The structure neck diameter D_{AN} may be at least one of 5%, 20%, 30%, 40%, 50%, 100%, 150%, 200%, or 300% greater than the neck diameter D_{N} of the second medicant container 302.

For example, the structure neck diameter D_{AN} may be any one of 13 mm or 20 mm. The structure neck diameter D_{AN} may be the same as a corresponding portion of an interface in the chamber 36 and/or the neck diameter of the first medicant container 34, or within +/−5%, 4%, 3%, 2%, or 1% of the diameter of the corresponding portion of the interface and the neck diameter of the first medicant container 34.

The assembled container structure 300 may define a main structure body diameter D_{A} that is greater than the main body diameter D_{M} of the second medicant container 302. The main structure body diameter D_{A} may be at least one of 10%, 20%, 30%, 40%, 50%, 100%, 150%, 200%, or 300% greater than the main body diameter D_{M} of the second medicant container 302.

For example, the main structure body diameter D_{A} may be any one of 16 mm, 22 mm, 24 mm, or 30 mm. The main structure body diameter D_{A} may be the same as the chamber diameter D_{C} and the main body diameter D_{M} of the first medicant container 34, or within +/−5%, 4%, 3%, 2%, or 1% of the chamber diameter D_{C} and the main body diameter D_{M} of the first medicant container 34.

The structure height H_{A} may be at least one of 10%, 20%, 30%, 40%, 50%, 100%, 150%, 200%, or 300% greater than the medicant container height H_{M} of the second medicant container 102.

The structure height H_{A} may be any one of 35 mm, 40 mm, 45 mm, 55 mm, 60 mm, 65 mm, or 75 mm. The structure height H_{A} may be at least one of 10%, 20%, 30%, 40%, 50%, 100%, 150%, 200%, or 300% greater than the chamber height H_{C}, such that the assembled container structure 300 extends outside of the chamber 36. The container structure 300 having such a height that it extends outside of the chamber 36 during use may provide for tamper resistance, as it may be more difficult for a user to inadvertently touch the plunger 250. In another embodiment, the structure height H_{A} is substantially the same as the medicant container height H_{M} of the first medicant container 34 or the chamber height H_{C}.

The head may include the head gasket 324 and a crimp 256 that is configured to secure the head gasket 324 to the lip 324. For example, the crimp 256 may have a cylindrical sidewalls that are configured to deform radially inwardly to attach to the bottom side of the lip 324. The crim 324 may include a central hole at the top to provide access, for example, for the inlet needle 70 to pierce the head gasket 324 to reach the medication inside the variable volume chamber 252.

The shell 304 may not define an internal support surface longitudinally between the bottom of the shell 304 and the cartridge body 312. For example, the sidewall 349 may define an interior cylindrical hole that extends from the base of the bottom end of the shell to the radially inwardly extending rim 352. Providing a hole between the plunger 250 and atmosphere can prevent a vacuum from forming in a space defined by the plunger 250 and the shell 304, for example, when the plunger 250 moves longitudinally toward the head 310.

The hole may provide sufficient space for a user's finger to actuate the plunger 250. In an embodiment an actuator (e.g., an electromechanical actuator) is provided to press the bottom of the plunger to move the plunger and thereby dispense medication.

Injecting medicant from the assembled container structure 300 may be substantially the same as with the first medicant container 34. For example, during the injection, the pump 72 may suction medication from the inlet needle 70 in the second medicant container 302 via the fluid line 74, and output the medication to the injection needle 38 for injection into the patient. As the plunger 250 moves during dispensing of the medication to equalize pressure within the variable volume chamber 252 and atmosphere, pressure within the shell 304 below the plunger 250 may equalize with atmosphere through the bottom hole in the shell 304.

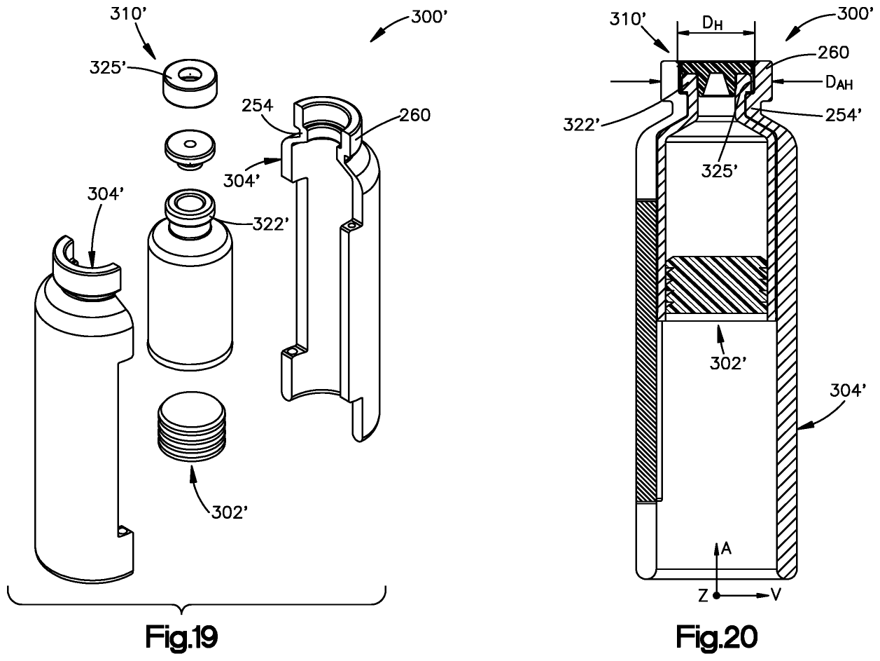

Referring now to FIGS. 19 and 20, another embodiment of the assembled container structure is shown. It is to be appreciated that this embodiment can be similar to the other embodiments of the assembled container structure shown, for example, in FIGS. 17 and 18. Accordingly, the same reference numbers used above with reference to features of the other embodiments can be also used with a “prime” notation in reference to similar features of this embodiment. It is also to be appreciated that, unless otherwise set forth below, the components (and features thereof) of the assembled container structure 300′ of this embodiment can be similar to those of the other embodiments.

The assembled container structure 300′ includes a second medicant container 302′ and a shell 304′. The shell 304′ may include a wall 254′ and a structure head wall 260 that extends longitudinally from the wall 254′ and may define an outer dimension that is intermediate an outer dimension defined by the wall 254′ and an outermost dimension defined by the shell 304′.

The structure head wall 260 may be radially outward of a lip 322′ of the second medicant container 302′ and extend circumferentially around the lip 322′. For example, the structure head wall 260 may radially abut a head 310′ of the second medicant container 302′. The head 310′ may include a crimp 325′, and a cylindrical sidewall of the crimp 325′ may radially abut the structure head wall 260 such that the second medicant container 302′ is secured from radial movement relative to the shell 304′.

The top end of the assembly container structure 300′ may define a medicant container interface that is configured to be received by the corresponding mechanical interface 54 of the injector 32. For example, the head 310′ and the structure head wall 260 may together define the medicant container interface that is configured to be received by the mechanical interface of the injector 32.

For example, the structure head wall 260 may define a structure head diameter D_{AH} that is substantially equal to a diameter of the cylindrical opening of the mechanical interface 54. For example, the structure head diameter D_{AH} may be any one of 7.5 mm, 13 mm, or 20 mm. In an embodiment, the structure head diameter D_{AH} is anywhere from 7.8 mm to 8.0 mm, anywhere from 13.3 mm to 13.5 mm, or anywhere from 20.3 mm to 20.5 mm. In an embodiment, the diameter of the cylindrical opening is the same as the structure head diameter D_{AH} or within +/−5%, 4%, 3%, 2%, or 1% of the structure head diameter D_{AH}.

The structure head diameter D_{AH} may be the same as the head diameter D_{H} of the first medicant container 34 or within +/−5%, 4%, 3%, 2%, or 1% of the head diameter D_{H} of the first medicant container 34.

The head 310′ may define a medicant container interface end that is undersized for the mechanical interface 54. The structure head diameter D_{AH} of the second medicant container 302′ may be at least one of 10%, 20%, 30%, 40%, 50%, 100%, 150%, 200%, or 300% greater than the head diameter D_{H} of the second medicant container 302′.

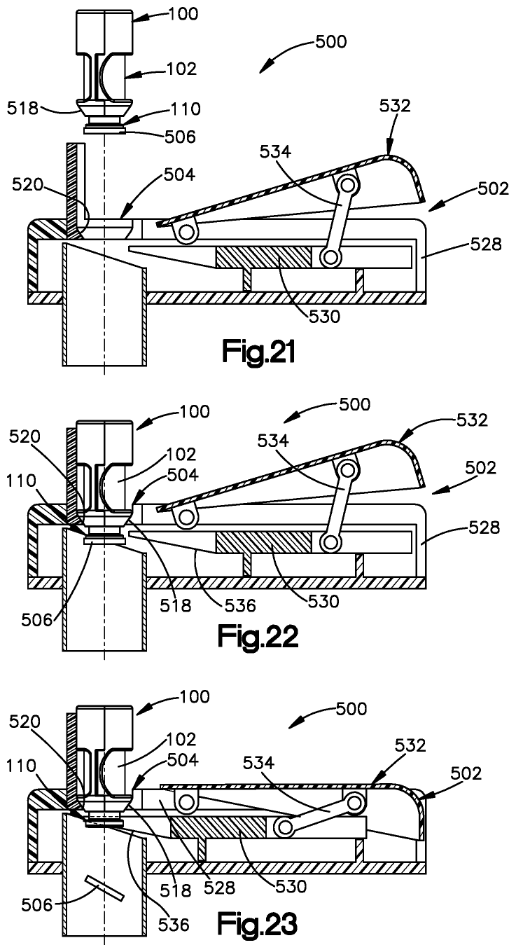

Referring now to FIGS. 21-23, use of a decapping system 500 (an example of a system) is progressively shown. The decapping system 500 may include a decapper 502 (an example of a device) and the assembled container structure 100, discussed above with reference to FIGS. 3-6.

The decapper 502 includes a decapping chamber 504 that may be configured to receive the assembled container structure 100 such that the decapper 502 is configured to remove a cap 506 from the assembled container structure 100. For example, the decapper 502 may be configured to remove the cap 506 from the head 110 of the second medicant container 102.

The decapping chamber 504 may define a decapper interface that is configured to receive a corresponding interface of the assembled container structure 100. For example, the assembled container structure 100 may include a radially inwardly extending shoulder 518 that defines an outwardly facing frusto-conically shaped surface and the decapper interface may define an inwardly facing frusto-conically shaped surface 520. When the outwardly facing frusto-conically shaped surface of the radially inwardly extending shoulder 518 abuts the inwardly facing frusto-conically shaped surface 520, the cap 506 may be disposed in a position such that the decapper 502 is configured to remove the cap 506.

The decapper 502 may include a housing 528, a movable member 530 that is movable relative to the housing 528, a handle 532 pivotable relative to the housing 528, and a linkage 534 pinned to the handle 532 and the movable member 530. The movable member 530 and linkage 534 are configured such that depression of the handle from an initial position in FIG. 21 to an actuated position in FIG. 23 actuates the movable member 530 toward the cap 506.

As shown in FIGS. 22 and 23, when the assembled container structure 100 is nested in the decapping chamber 504 and the handle 532 is actuated, the movable member moves toward the cap 506 such that a ramped surface 536 urges the cap 506 away from the head 110. For example, the ramped surface 536 may be bifurcated or formed by a separate movable member (e.g., disposed in front of the page when viewing FIGS. 22 and 23) such that the ramped surface 536 (and/or separate ramped surface) engages the top (when viewing FIGS. 22 and 23) of the cap 506 at two circumferentially spaced apart positions to urge the cap 506 downward away from the head 110, thereby removing the cap 506 as shown in FIG. 23.

The head 110 may define a decapper interface that is undersized such that, for example, the decapper 502 would not be configured to remove the cap 506 from the head 110 when only the second medicant container 102 is disposed in the decapping chamber 504. For example, when only the second medicant container 102 is disposed within the decapping chamber 504, the cap 506 may be disposed too low for the decapper 502 to remove the cap 506. The ramped surface 536 may thus be too high to remove the cap 506, when only the second medicant container 102 is disposed in the decapping chamber 504.

In some embodiments, any of the other assembled container structures are configured to nest in the chamber of the decapper such that the decapper is configured to remove a cap from the assembled container structure. The first medicant container 34 (shown in FIGS. 1 and 2) may be configured to nest in the decapping chamber 504 in a similar manner as the assembled container structure 100, such that the decapper 502 would be configured to remove the cap 506 from the head 56.

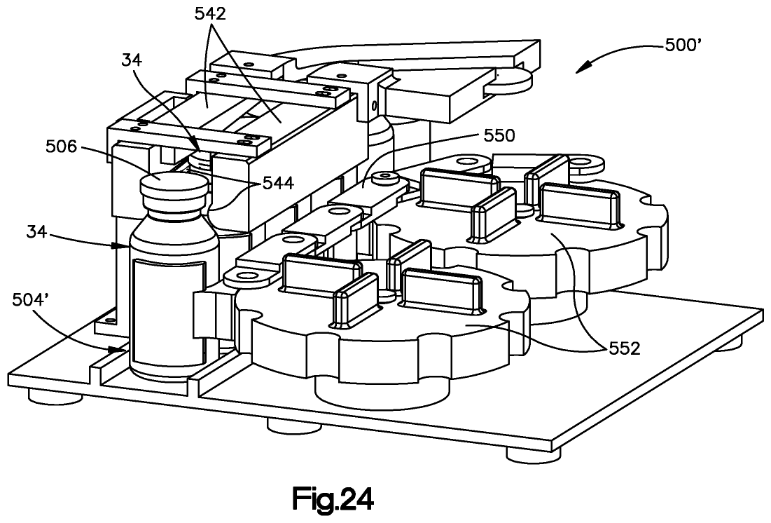

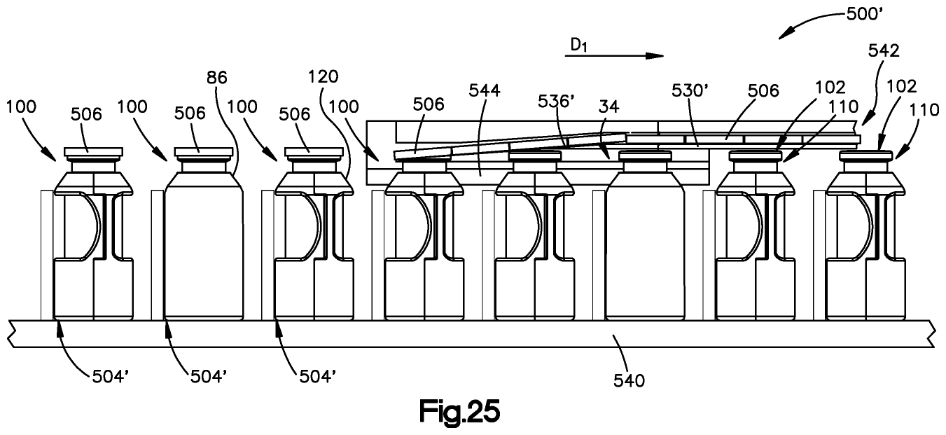

Referring now to FIGS. 24 and 25, another embodiment of the decapping system is shown. It is to be appreciated that this embodiment can be similar to the embodiment of the decapping system shown in FIGS. 21-23. Accordingly, the same reference numbers used above with reference to features of the other embodiments can be also used with a “prime” notation in reference to similar features of this this embodiment. It is also to be appreciated that, unless otherwise set forth below, the components (and features thereof) of the assembled container structure 500′ of this embodiment can be similar to those of the other embodiments.

The decapping system 500′ (an example of a system) may include a plurality of decapping chambers 504′ that are each configured to selectively receive the first medicant container 34 or the assembled container structure 100 to remove a corresponding cap 506′ from each of a plurality of first medicant containers 34 and assembled container structures 100. For example, the decapping chambers 504′ may be moveable in directed D₁ relative to a platform 540. In some embodiments, the decapping chambers 504′ may be fixed relative to the platform, which may be a conveyor that is configured to move the decapping chambers 504′ in a first direction D1.

The decapping system 500′ may include opposing rail members 542 that each define a respective fixed member 530′ (e.g., as illustrated in FIG. 25) that are symmetrical and diametrically oppose one another. Turning now to FIG. 25, the fixed member 530′ includes a ramped surface 536′ that is configured to urge the caps 506 away from the corresponding first medicant container 34 or assembled container structure 100, when the corresponding first medicant container 34 or assembled container structure 100 is nested in the decapping chamber 504′. For example, as the decapping chambers 540′ are moved in the first direction D1, the caps 506 move to the left (when viewing FIG. 25) of the ramped surface 536′ move in the first direction D1 toward the ramped surface 536′ such that the ramped surface 536′ urges the caps 506 away from the corresponding first medicant container 34 and assembled container structures 100.

For example, the ramped surfaces 536′ may be formed by separate rail members 542 (e.g., one rail member 542 is disposed in front of the page when viewing FIG. 24) such that the ramped surfaces 536′ (and/or separate ramped surface) engages the bottom (when viewing FIG. 25) of each cap 506 at two circumferentially spaced apart positions to urge each cap 506 upward away from the corresponding first medicant container 34 and assembled container structures 100. Urging each cap 506 in such a manner may result in removing each cap 506 as shown with regard to the first medicant container 34 and assembled container structures 100 that are spaced in the first direction from the start of the ramped surface 536′. In an embodiment, the ramped surfaces are formed by a single bifurcated member.

The head 110 may define a decapper interface that is undersized such that, for example, the decapper 502′ would not be configured to remove a cap 506 from the head 110 when only the second medicant container 102 is disposed in the decapping chamber 504. For example, when only the second medicant container 102 is disposed within one of the decapping chambers 504′, the corresponding cap 506 may be disposed too low for the decapper 502′ to remove the cap 506. The ramped surface 536′ may thus be too high to remove the cap 506, when only the second medicant container 102 is disposed in the decapping chamber 504′.

In some embodiments, any of the other assembled container structures are configured to nest in the chamber of the decapper such that the decapper is configured to remove a cap from the assembled container structure.

Each rail member 542 may include a protrusion 544 that is configured to abut the radially inwardly extending shoulders 86 and 518 of the first medicant containers 34 and the assembled container structures 100. For example, as the first medicant containers 34 and the assembled container structures 100 move in the first direction Di such that the ramped surfaces 536′ urge each cap 506 upwards, each protrusion 544 may abut the radially inwardly extending shoulders 86 and 518 of the corresponding first medicant containers 34 and assembled container structures 100. The protrusions 544 abutting the radially inwardly extending shoulders 86 and 518 of the corresponding first medicant containers 34 and assembled container structures 100 may prevent the corresponding first medicant containers 34 and assembled container structures 100 from moving upward with the corresponding caps 506 as the caps 506 are being removed.

The protrusions 544 may be configured to allow the first medicant containers 34 and assembled container structures 100 to move along the protrusions 544 in the first direction D₁. For example, the protrusions 544 may be configured to not abut any part of the first medicant containers 34 and assembled container structures 100, unless the first medicant containers 34 and assembled container structures 100 are moved upward from the platform 540 (e.g., as the corresponding cap 506 is being removed).

Referring again to FIG. 24, the decapping system 500′ may include an actuation chain 550 that is fixed relative to the decapping chambers 504′ and in mesh with a plurality of gears 552. One or more of the gears 552 may be configured to actuate the chain 550 such that the decapping chambers 504′ move in the first direction D₁. For example, an actuator (not shown), such as a motor, may be configured to rotate one or more of the gears 552 to actuate the chain 550 to move the decapping chambers 504′ move in the first direction D₁.

Certain terminology is used in the following description for convenience only and is not limiting. The words “right”, “left”, “lower” and “upper” designate directions in the drawings to which reference is made. The words “inner”, “internal”, and “interior” refer to directions towards the geometric center of the assembled container structure, while the words “outer”, “external”, and “exterior” refer to directions away from the geometric center of the assembled container structure. The words, “anterior”, “posterior”, “superior,” “inferior,” “medial,” “lateral,” and related words and/or phrases are used to designate various positions and orientations in the human body to which reference is made. When these words are used in relation to the system or a component thereof, they are to be understood as referring to the relative positions of the system, for example, as a patch pump attached to a human body. The terminology includes the above-listed words, derivatives thereof and words of similar import.

1. A system comprising:
a device including a chamber configured to receive a first medicant container having a first size configured to nest in the chamber;
a second medicant container having a second size that is smaller than the first size, such that the second medicant container is not configured to nest in the chamber; and
a shell configured to cover at least a portion of the second medicant container so as to define an assembled container structure that is configured to nest in the chamber.

2. The system of claim 1, wherein the system is an injector system that comprises:
an injector including the chamber, wherein the chamber is configured to receive the first medicant container such that the injector is configured to inject contents of the first medicant container, and whereby the injector is configured to inject the contents of the second medicant container when the assembled container structure is disposed within the chamber.

3. The system of claim 1, wherein the system is a decapping system that comprises:
a container decapper including the chamber, wherein the chamber is configured to receive the first medicant container such that the container decapper is configured to remove a first cap from the first medicant container, whereby the container decapper is not configured to remove a second cap from the second medicant container when only the second medicant container is received in the chamber, and whereby the container decapper is configured to remove the second cap from the second medicant container when the assembled container structure is disposed within the chamber.

4. The system of claim 1, wherein when the assembled container structure is nested in the chamber, the chamber has a cross-sectional dimension, perpendicular to a longitudinal axis that the assembled container structure and the chamber extend along, that is substantially equal to a cross-sectional dimension of the assembled container structure.

5-10. (canceled)

11. The system of claim 1, wherein the shell comprises a radially inwardly extending rim and a floor that is offset from the radially inwardly extending rim along a longitudinal axis that the assembled container structure extends along, wherein the radially inwardly extending rim is configured to abut a shoulder of the second medicant container, and wherein the floor is configured to abut an end of the second medicant container opposite the shoulder along the longitudinal axis.

12-16. (canceled)

17. The system of claim 1, wherein a side wall of the assembled container structure is defined by the shell.

18. The system of claim 1, wherein the shell comprises a pair of interlocking bodies that when interlocked together define a receptacle that is configured to hold the second medicant container relative to the shell, such that the assembled container structure is defined when the receptacle holds the second medicant container.

19-20. (canceled)

21. The system of claim 1, wherein the shell further comprises:
an outer surface having at least one outer dimension that is substantially equal to a corresponding at least one outer dimension of a first standard size of a standard, wherein the outer surface is configured to be received in the chamber, of the device, which corresponds to the first standard size; and
an internal cavity having at least one inner dimension that is substantially equal to a corresponding at least one inner dimension of a second standard size of the standard, the second standard size being less than the first standard size, and wherein the internal cavity is configured to receive a container that corresponds to the second standard size.

22-23. (canceled)

24. The system of claim 1, wherein the shell includes a window, is transparent, and/or is translucent such that a main body of the second medicant container is at least partially visible through the shell when the shell covers at least the portion of the second medicant container so as to define the assembled container structure.

25. (canceled)

26. The system of claim 1, wherein the shell includes a radially outer wall that extends circumferentially around a lip of the second medicant container.

27. The system of claim 1, further comprising:
a kit comprising:
the first medicant, the first medicant container containing a medication;
the second medicant container, the second medicant container containing the medication; and
the shell configured to receive the second medicant container so as to define
the assembled container structure,
wherein the assembled container structure has outer dimensions that are substantially equal to outer dimensions of the first medicant container.

28. The system of claim 27, wherein the assembled container structure has a cross-sectional dimension, perpendicular to a longitudinal axis that the assembled container structure extends along, that is substantially equal to a cross-sectional dimension of the first medicant container, and wherein the second medicant container has a cross-sectional dimension perpendicular to the longitudinal axis that is less than the cross-sectional dimensions of the first medicant container and the assembled container structure.

29-32. (canceled)

33. An assembled container structure comprising:
a medicant container defining a base, a side wall extending up from the base to a neck, and a medicant container interface end extending up from the neck, wherein the medicant container contains a medication; and
a shell configured to receive the medicant container so as to define the assembled container structure that is sized in cross-section greater than the medicant container,
wherein the shell has an internal support surface that supports the base of the medicant container, and the internal support surface is spaced from the base of the shell so as to define a gap therebetween,
wherein the shell extends to the neck of the medicant container and terminates at the neck, such that the medicant container interface end of the medicant container extends out from the shell, and
wherein the assembled container structure is configured to interface with an injector.

34-36. (canceled)

37. The assembled container structure of claim 33, further comprising a support structure that extends in the gap from the base of the shell to the base of the medicant container, and wherein the gap is partially defined by the side wall of the shell.

38-39. (canceled)

40. The assembled container structure of claim 33, wherein the medicant container defines a height, comprises a head defining a head diameter, and comprises a main body portion defining a main body diameter, and wherein i) the main body diameter is 22 mm, the head diameter is 20 mm, and the height is 45 mm, ii) the main body diameter is 22 mm, the head diameter is 20 mm, and the height is 40 mm, or iii) the main body diameter is 16 mm, the head diameter is 13 mm, and the height is 45 mm; and
wherein the assembled container structure defines a main body diameter, defines a structure head diameter, and defines a height, and wherein i) the main body diameter is 24 mm, the structure head diameter is 20 mm, and the height is 45 mm, ii) the main body diameter is 24 mm, the structure head diameter is 20 mm, and the height is 60 mm, iii) the main body diameter is 30 mm, the structure head diameter is 20 mm, and the height is 55 mm, or iv) the main body diameter is 30 mm, the structure head diameter is 20 mm, and the height is 60 mm.

41. (canceled)

42. The assembled container structure of claim 33, wherein at least a portion of the shell is colored, tinted, or opaque such that a user is unable to determine the color of medicant held by the medicant container.

43. A shell comprising:
an outer surface having at least one outer dimension that is substantially equal to a corresponding at least one outer dimension of a first size of a standard, wherein the outer surface is configured to be received in a chamber of a device that corresponds to the first size; and
an internal cavity having at least one inner dimension that is substantially equal to a corresponding at least one inner dimension of a second size of the standard, the second size being less than the first size, and wherein the internal cavity is configured to receive a container that corresponds to the second size.

44. The shell of claim 43, wherein the standard is ISO 8362-1.

45. The shell of claim 44, wherein the first size is one of 4R, 6R, 8R, 10R, 15R, 20R, 25R, or 30R, and wherein the second size is one of 2R, 4R, 6R, 8R, 10R, 15R, 20R, or 25R.

46. (canceled)

47. A kit comprising:
the shell of claim 44, which is a first shell; and
a second shell, wherein the first shell and the second shell have different ones of the at least one inner dimension and the at least one outer dimension, and wherein none of the outer dimensions have a minimum corresponding dimension of the standard, and wherein none of the inner dimensions have a maximum corresponding dimension of the standard.
